(19) United States
(12) Patent Application Publication
(10) Pub. No.: US 2025/0154213 A1
(43) Pub. Date: May 15, 2025

(54) De Novo Designed Cortisol Biosensor

(71) Applicant: University of Washington, Seattle, WA (US)

(72) Inventors: Hsien-Wei YEH, Seattle, WA (US); David BAKER, Seattle, WA (US)

(21) Appl. No.: 18/945,961

(22) Filed: Nov. 13, 2024

Related U.S. Application Data
(60) Provisional application No. 63/599,104, filed on Nov. 15, 2023.

(51) Int. Cl. C07K 14/47; A61K 38/00; G01N 33/53; G01N 33/74

(52) U.S. Cl. CPC C07K 14/47; G01N 33/53; G01N 33/743; A61K 38/00; C07K 2319/70; G01N 2333/575

(57) ABSTRACT

Polypeptides are disclosed having an amino acid sequence at least 50% identical to the amino acid sequence of SEQ ID NO:1, wherein, relative to SEQ ID NO:1, residue 43 is I, residue 95 is Q, and residue 128 is L, fusion proteins thereof, kits thereof, and methods for using the polypeptides for treating a disorder associated with cortisol, or for detecting cortisol in a biological sample.

### FEDERAL FUNDING STATEMENT

This invention was made with government support under Grant No. 1 K99 EB 031913-01A1, awarded by the National Institute of Biomedical Imaging and Bioengineering. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Oct. 30, 2024 having the file name “23-1607-US.xml” and is 25,122 bytes in size.

### BACKGROUND

The design of small molecule binding proteins with high affinity and specificity is of considerable interest. For example, biosensors and switches that undergo dimerization upon ligand binding (chemically-induced dimerization (CID)) are broadly useful, but current approaches focus on engineering natural CID systems as general methods are not currently available for designing protein small molecule interactions and linking these to protein association.

### SUMMARY

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1, wherein, relative to SEQ ID NO:1, residue 43 is I, residue 95 is Q, and residue 128 is L. In one embodiment, the polypeptides comprises an amino acid sequence at least 75% identical to SEQ ID NO:1. In another embodiment, the polypeptides comprises an amino acid sequence at least 90% identical to SEQ ID NO:1. In a further embodiment, the polypeptide comprises an amino acid sequence at least 95% identical to SEQ ID NO:1.

In one embodiment, substitutions relative to SEQ ID NO:1 are selected from the residues shown in the substitution column on Table 1.

In one embodiment, relative to SEQ ID NO:1, the polypeptide is identical at 1, 2, 3, 4, 5, 6, 7, 8, 9, 10, 11, 12, 13, 14, or all 15 of the identified residues that interact with cortisol. These residues are identified in the far right column of Table 1. In another embodiment, substitutions relative to the SEQ ID NO:1 are conservative amino acid substitutions.

The disclosure also provides fusion proteins, comprising the polypeptide of any embodiment or combination of embodiments of this first aspect, fused to one or more functional domains.

In a second aspect, the disclosure provides polypeptides comprising an amino acid sequence at least 50%, 55%, 60%, 65%, 70%, 75%, 80%, 85%, 90%, 95%, 98%, or 100% identical to the amino acid sequence selected from SEQ ID NO:2-21. In one embodiment, the polypeptide comprises an amino acid sequence at least 75% identical to the amino acid sequence selected from SEQ ID NO:2-21. In another embodiment, the polypeptide comprises an amino acid sequence at least 90% identical to the amino acid sequence selected from SEQ ID NO:2-21. In a further embodiment, the polypeptide comprises an amino acid sequence at least 95% identical to the amino acid sequence selected from SEQ ID NO:2-21.

In one embodiment, relative to the reference amino acid sequence, the polypeptide is identical at 1, 2, 3, 4, 5, 6, 7, 8, or all of the identified residues that form an interface with cortisol and the polypeptide of embodiment of the first aspect of the disclosure. In another embodiment, substitutions relative to the reference sequence are conservative amino acid substitutions.

In another embodiment of this second aspect, the disclosure provides fusion proteins comprising the polypeptide of any embodiment of the second aspect fused to one or more functional domains. In various non-limiting embodiments, the functional domain may comprise, for example, a targeting domain, a detectable domain, a scaffold domain, a secretion signal, an Fc domain, or a further therapeutic peptide domain. In one embodiment, the functional domain comprises a first domain of a detectable protein that can be complemented by a second domain of the detectable domain when brought into proximity by, for example, cortisol induced dimerization of a first polypeptide of the first aspect of the disclosure, and a second polypeptide according to this second aspect of the disclosure. In one embodiment, the first domain and second domains of the detectable protein comprise first and second domains of a luciferase protein.

In another aspect, the disclosure provides nucleic acids encoding the polypeptide or fusion protein of any embodiment or combination of embodiments of the disclosure. In a further aspect, the disclosure provides expression vectors comprising the nucleic acid of any aspect of the disclosure operatively linked to a suitable control sequence, such as a promoter. In another aspect, the disclosure provides host cells that comprise the polypeptide, fusion protein, nucleic acid or expression vector (i.e.: episomal or chromosomally integrated) disclosed herein, wherein the host cells can be either prokaryotic or eukaryotic.

The disclosure also provides pharmaceutical compositions, comprising:
(a) the polypeptide, fusion protein, nucleic acid, expression vector, and/or host cell of any embodiment or combination of embodiments herein; and
(b) a pharmaceutically acceptable carrier.

In another embodiment, the disclosure provides kits, comprising:
(a) one or more first polypeptides or fusion proteins according to any embodiment or combination of embodiments of the first aspect of the disclosure, or a nucleic acid/expression vector encoding the first polypeptide or fusion protein; and
(b) one or more second polypeptides or fusion proteins according to any embodiment or combination of embodiments of the second aspect of the disclosure, or a nucleic acid/expression vector encoding the second polypeptide or fusion protein.

In one embodiment,
(i) the one or more first polypeptides or fusion proteins comprise a fusion protein of the first aspect of the disclosure, wherein the one or more functional domains comprises a first domain of a detectable protein; and
(ii) the one or more second polypeptides or fusion proteins comprise a fusion protein of the second aspect of the disclosure, wherein the one or more functional domains comprises a second domain of the detectable protein;
wherein the first domain of the detectable protein and the second domain of the detectable protein are only detectable when brought into proximity by formation of a chemically-induced dimer complex comprising cortisol, the first fusion protein, and the second fusion protein.

In one embodiment, the detectable protein comprises first and second luciferase domains. Exemplary first and second luciferase domains comprise SEQ ID NO:22 and 23. In another embodiment, the fusion proteins comprise an amino acid sequence at least 50%, 55%, 60%, 65%, 70%, 75%, 80%, 85%, 90%, 95%, 98%, or 100% identical to the amino acid sequence selected from SEQ ID NO:24 and 25.

In other embodiments of the kit of any embodiment herein, the kit may further comprise one or more of:
(i) cortisol; and/or
(ii) a means for detection of the reporter protein, including but not limited to a luciferase substrate.

In another aspect, the disclosure provides methods for treating a disorder associated with cortisol, comprising administering to a subject in need thereof an amount effective to treat the disorder of the polypeptide or fusion protein of any embodiment or combination of embodiments of the first aspect of the invention; or a nucleic acid, expression vector, host cell, and/or pharmaceutical composition thereof. In various embodiments, the disorder is selected from the group consisting of Cushing's syndrome and Addison's disease.

In another aspect, the disclosure provides methods for detecting cortisol in a biological sample, comprising
(a) contacting the biological sample with an amount of the polypeptide or fusion protein of any embodiment or combination of embodiments of the first aspect of the disclosure effective bind to cortisol in the biological sample to produce a binding complex, and
(b) detecting the binding complex in the biological sample, thereby detecting cortisol in the biological sample.

In another embodiment, the methods comprise
(a) contacting the biological sample with:
   (i) one or more first polypeptides or fusion proteins any embodiment or combination of embodiments of the first aspect of the disclosure; and
   (ii) one or more second polypeptides or fusion proteins any embodiment or combination of embodiments of the second aspect of the disclosure; and
(b) detecting a ternary binding complex in the biological sample between (i) the first polypeptide or fusion protein, (ii) the second polypeptide or fusion protein, and (iii) cortisol present in the biological sample, thereby detecting cortisol in the biological sample.

In one embodiment,
(i) the one or more first polypeptides or fusion proteins comprise a fusion protein, wherein the one or more functional domains comprises a first domain of a detectable protein; and
(ii) the one or more second polypeptides or fusion proteins comprise a fusion protein, wherein the one or more functional domains comprises a second domain of the detectable protein;
wherein the first domain of the detectable protein and the second domain of the detectable protein are only detectable upon formation of the ternary complex.

In another embodiment, the detectable protein may comprise, but is not limited to, luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease). In one embodiment, the detectable protein comprises a luciferase

### DESCRIPTION OF THE FIGURES

FIG. 1. The NTF2 fold has a designable fold made up of ideal secondary structure elements (left panel), the large pocket (middle panel) of NTF2s can bind various small-molecules, and the binding mode of small-molecules in NTF2s enables the generation of CID sensors (right panel). (B) Design pipeline for small-molecule-binding in the NTF2 fold.

FIG. 2. Design and characterization of a cortisol-dependent heterodimer. (A) Design pipeline for cortisol-induced heterodimerization. Starting from a model of seq129.1_CID in complex with cortisol (top), we used RIFdock™ and a library of designed 3-helix bundles to generate thousands of putative ternary complexes (middle), and amino acid sequences encoding these complexes were generated with FastDesign™ and ProteinMPNN™ (bottom). Zoom-in of the designed CID ternary complex (seq129.1_CID, mini11, and cortisol) (bottom panel). (B) Size-exclusion chromatography traces of an equimolar solution of seq129.1 and mini11 (1 μM) in the presence or absence of cortisol (10 μM). (C) Schematic diagram of the designed cortisol-induced heterodimerization coupled with a binary split luciferase to create a biosensor. (D) Luminescent response of an equimolar solution (200 nM) of seq129.1_CID-SmBiT and mini11_LgBiT titrated with cortisol.

### DETAILED DESCRIPTION/CLAIMS

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), Dang, B. et al. SNAC-tag for sequence-specific chemical protein cleavage. Nat. Methods 16, 319-322 (2019), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Any N-terminal methionine residue in any polypeptide of the disclosure may be present or may be deleted. In all embodiments of the polypeptides disclosed herein, 1, 2, 3, 4, or 5 residues may be deleted from the N-terminus and/or the C-terminus of the polypeptide while retaining activity.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1, wherein, relative to SEQ ID NO:1, residue 43 is I, residue 95 is Q, and residue 128 is L.

(SEQ ID NO: 1)
TSSKEAEEAIRDMLRRWYEAINKGDMEKLKSLVDPDASFHFAITNQQYD
KEQFLEMIKEALKQDLKVEVKSIHIQQQPRGDHVTVTVHVEAHMNQNGQ
THTFTVTDHYHFVRKGDSWKITRTQWHIHLQ

The polypeptides of this aspect of the disclosure bind cortisol with micromolar or nanomolar affinity, and thus can be used in therapeutic and diagnostic methods as described herein. The polypeptides of this aspect may also be used in as part of a cortisol biosensor as described herein.

In one embodiment, the polypeptides comprises an amino acid sequence at least 75% identical to SEQ ID NO: 1. In another embodiment, the polypeptides comprises an amino acid sequence at least 90% identical to SEQ ID NO: 1. In a further embodiment, the polypeptide comprises an amino acid sequence at least 95% identical to SEQ ID NO: 1.

In one embodiment, substitutions relative to SEQ ID NO:1 are selected from the residues shown in the substitution column on Table 1. As used in Table 1, polar residues are D, E, H, K, N, Q, R, S, and T, while nonpolar residues are A, F, G, I, L, M, P, V, W, and Y. The substitutions are based on site saturation mutagenesis studies (data not shown).

TABLE 1

| Residue | Starting | | |
|---|---|---|---|
| # | scaffold | substitutions | key_feature |
| 1 | T | polar | |
| 2 | S | polar | |
| 3 | S | polar | |
| 4 | K | polar | |
| 5 | E | polar | |
| 6 | A | T, V | |
| 7 | E | polar, A | |
| 8 | E | polar | |
| 9 | A | M, H | |
| 10 | I | G, V | |
| 11 | R | polar, I | |
| 12 | D | polar, A | |
| 13 | M | E | |
| 14 | L | P, G, A, V, I, S, | |
| | | T, Q, E | |
| 15 | R | I, V, G, P, polar | |
| 16 | R | V, P, polar | |
| 17 | W | Y | interacts with cortisol |
| 18 | Y | H, F | HBNet residue |
| 19 | E | polar | |
| 20 | A | Y, E | |
| 21 | I | P, V, I, L, T, Q, | interacts with cortisol |
| | | E, F | |
| 22 | N | P | structural HBNet |
| 23 | K | P, polar | |
| 24 | G | I, W, S, D, E | |
| 25 | D | M, polar | |
| 26 | M | N, A, polar | |
| 27 | E | V, G, polar | |
| 28 | K | W, polar | |
| 29 | L | I, V | |
| 30 | K | P, polar | |
| 31 | S | A, polar | |
| 32 | L | I | |
| 33 | V | I, M, W, D | |
| 34 | D | E, N | |
| 35 | P | polar | |
| 36 | D | A, polar | |
| 37 | A | E, S, Y, W, V | |
| 38 | S | T, polar | |
| 39 | F | W, Y, L | |
| 40 | H | I, V, polar | |
| 41 | F | P, A, C, V, I, L, | interacts with cortisol |
| | | M, D, W, Y, E | |
| 42 | A | N, polar | |
| 43 | I | None | Interface with proteins of SEQ |
| | | | ID NO:2-21 and dependent |
| | | | claims for cortisol biosensor |
| 44 | T | A, N, polar | |
| 45 | N | H, K, polar | |
| 46 | Q | C, F, W, K | |
| 47 | Q | A, I, M, Y, T, K, V | |
| 48 | Y | P, C, L | |
| 49 | D | C | |
| 50 | K | L | |
| 51 | E | P, polar | |
| 52 | Q | polar | |
| 53 | F | C, L, Y, W | interacts with cortisol |
| 54 | L | None | |
| 55 | E | polar | |
| 56 | M | I, Q, Y | interacts with cortisol |
| 57 | I | N, E, V | |
| 58 | K | polar | |
| 59 | E | I, W, D, polar | |
| 60 | A | E, L | |
| 61 | L | P, H | |
| 62 | K | polar | |
| 63 | Q | V, F, Y,H | near cortisol interface |
| 64 | D | E, polar | |
| 65 | L | S, A | interacts with cortisol |
| 66 | K | I, polar | |
| 67 | V | none, (Sam: A, L, I) | interacts with cortisol |
| 68 | E | polar | |
| 69 | V | Y | |
| 70 | K | V, P, L, polar | |
| 71 | S | A, polar | |
| 72 | I | M, F | |
| 73 | H | L, Y, polar | |
| 74 | I | A, F, T | |
| 75 | Q | A, M, polar | |
| 76 | Q | L, K, polar | |
| 77 | Q | H, polar | |
| 78 | P | A, V, W, T, H, polar | |
| 79 | R | H, F, C, polar | |
| 80 | G | P, V, polar | |
| 81 | D | H, Y, polar | |
| 82 | H | polar, V | |
| 83 | V | K | |
| 84 | T | P, I, polar | |
| 85 | V | A | |
| 86 | T | S, W, A, polar | |
| 87 | V | G, A, I, F, T | |
| 88 | H | I, S, N, D, polar | |
| 89 | V | G, L | near cortisol interface |
| 90 | E | G, polar | |
| 91 | A | L, M, T, D | interacts with cortisol |
| | | (sam: V, G), M | |
| 92 | H | polar | |
| 93 | M | V, E (sam : A, L, I), | |
| | | N | |
| 94 | N | polar, V | |
| 95 | Q | None | Interface with proteins of |
| | | | SEQ ID NO: 2-21 and |
| | | | dependent claims for |
| | | | cortisol biosensor |
| 96 | N | I, Y, K, polar | |
| 97 | G | A, C, K, polar | |
| 98 | Q | A, C, I, K, polar | |
| 99 | T | I, F, S, V, polar | |
| 100 | H | N, polar | |
| 101 | T | W, V, polar | |
| 102 | F | (sam: A, V, I, L, M, | interacts with cortisol |
| | | Y) | |
| 103 | T | A, L, R, K, polar | |
| 104 | V | C, I, F, Y, S | interacts with cortisol |
| 105 | T | Y, K, V, polar | |
| 106 | D | G, C, F, S, K | interacts with cortisol |
| 107 | H | P, V, R, polar | |
| 108 | Y | none | interacts with cortisol/HBNet |
| 109 | H | L, W, S, N, D, polar | |
| 110 | F | C, I, L, S, R | |
| 111 | V | D, polar | |
| 112 | R | polar (sam: A, M) | |
| 113 | K | Q, I, polar | |
| 114 | G | W, S, D, polar | |
| 115 | D | G, polar | |
| 116 | S | G, A, K, polar | |
| 117 | W | L, M, S, Q | |
| 118 | K | A, C, F, N, polar | |
| 119 | I | V, H, R | |
| 120 | T | I, K, V, polar | |
| 121 | R | polar | |
| 122 | T | G, A, C, V, I, M, W, | interacts with cortisol |
| | | S, T, N, Q, D, E, R, | |
| | | K | |
| 123 | Q | L, K, V, polar | |
| 124 | W | C, V, I, L, M, F , W, | interacts with cortisol |
| | | Y, N, Q, H, D, S | |
| 125 | H | Y, F, polar | |
| 126 | I | Y, D, R, K, T | interacts with cortisol |
| 127 | H | A, F, R, K, polar | |
| 128 | L | None | Interface with proteins of |
| | | | SEQ ID NO: 2-21 and |
| | | | dependent claims for |
| | | | cortisol biosensor |
| 129 | Q | polar, L | |

In one embodiment, relative to SEQ ID NO:1, the polypeptide is identical at 1, 2, 3, 4, 5, 6, 7, 8, 9, 10, 11, 12, 13, 14, or all 15 of the identified residues that interact with cortisol. These residues are identified in the far right column of Table 1.

In another embodiment, substitutions relative to the SEQ ID NO:1 are conservative amino acid substitutions.

As used throughout the disclosure, such conservative amino acid substitutions involve replacing a residue by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe.

The disclosure also provides fusion proteins, comprising the polypeptide of any embodiment or combination of embodiments of this first aspect, fused to one or more functional domains.

As used throughout the disclosure, any functional domain may be fused to the polypeptide. In various non-limiting embodiments, the functional domain may comprise, for example, a targeting domain, a detectable domain, a scaffold domain, a secretion signal, an Fc domain, or a further therapeutic peptide domain. In one embodiment, the functional domain comprises a first domain of a detectable protein that can be complemented by a second domain of the detectable domain when brought into proximity by, for example, cortisol induced dimerization of a first polypeptide of this first aspect of the disclosure, and a second polypeptide according to the second aspect of the disclosure (see below). In one embodiment, the first domain and second domains of the detectable protein comprise first and second domains of a luciferase protein. The functional domain(s) may be present as an insertion at a loop region of the polypeptides, and/or at one or both termini of the fusion protein.

In a second aspect, the disclosure provides polypeptides comprising an amino acid sequence at least 50%, 55%, 60%, 65%, 70%, 75%, 80%, 85%, 90%, 95%, 98%, or 100% identical to the amino acid sequence selected from SEQ ID NO:2-21. The polypeptides of this aspect can be used, for example, as part of a cortisol biosensor as described herein. The inventors have shown that the polypeptides of this aspect of the disclosure can be used as part of a cortisol-dependent dimerization system, together with the polypeptides of the first aspect of the disclosure (see above). The polypeptides of this second aspect of the disclosure bind to the polypeptides of the first aspect only in the presence of cortisol, resulting in cortisol-induced dimerization, as detailed in the examples that follow, and can thus be used as cortisol biosensors.

The amino acid sequence of SEQ ID NO:2-21 are shown in Table 2. The interface residues with the polypeptides of the first aspect of the disclosure are shown in bold font.

TABLE 2
>HHH_b1_04181_000000111_0001_A
SEEARLTLIVFRIAVEFGLSQEDYRRLFELARELLERGISLEEIEKILRKVAEELL (SEQ ID
NO: 2)
MSSMDKSKLTLKVFNIAMEYGLNDEQYQQLADFAFKKLEEGKSLEEIEKELREKAKELASSLE
(SEQ ID NO: 12)
>HHH_b1_04181_000000196_0001_A
DLEARLTLIVFRIAVQFGLSQEDYRRLAELARELLERGISLEEIEKILRKVAEELL (SEQ ID
NO: 3)
MSSMDKARMTFECFLIAMKYGLDDEKYQELCKLAFEMLDQGKSFEEIKKEFEEKAKELKSSLE
(SEQ ID NO: 13)
>HHH_b1_06494_000000186_0001_A
SKELLEILLRAAERIEDPKERRFLLEFAVESAEINGDEELLKRLRELLKKLK (SEQ ID NO: 4)
MSSSSGEELLKKLYEGAQTISDKKQKLFLLNFAKSIAEKHGEEELLKKIKEAIKELQSXSSLE
(SEQ ID NO: 14)
>HHH_b1_06682_000000007_0001_A
DREERLREAVEFLVKLLGLSEEQKEELERLVERLVEEGVDLAQALVILFALAIHL
(SEQ ID NO: 5)
MSSSDREKLKEYADILAEQAGLTEEQLEELKKEVDKLVAEGVPYTSALVQLFLKAVQLSSSLE
(SEQ ID NO: 15)
>HHH_b1_08005_000000095_0001_A
DEEFRRILEEAKKLIKHIPDPELRFFLEFLLRAVERSGNPKLLKALESAVKFVEKRL (SEQ ID
NO: 6)
MSDEELKEVVERAKELIEKITDPELKFFLSFLLKAAEESKKPSMIKQLKEAIDFVEKKQSSLE
(SEQ ID NO: 16)
>HHH_b1_08746_000000057_0001_A
SEDAEELFRRAEKLEEEGNIKKAQVVATLAWFSAAARKDEELLKRIEELVQRLYRLG (SEQ ID
NO: 7)
MSSPEAKKYYEEAKKLAEEGDYKKALVKATLALFLAQLDNDEELVEKTKELLEEIQKKRSSLE
(SEQ ID NO: 17)
>HHH_b2_00727_000000213_0001_A
TRTLVEVFLLLAQAARIDDPEEREKVLREAERVAKENNDPSAERLVELVERKLRN (SEQ ID
NO: 8)
MSSASTLVKTFSLCMEALSIEDPEKREEVYEKARKLAEENNDPAALFLVESIKKQHEQSSSLE
(SEQ ID NO: 18)
>HHH_b2_02091_000000170_0001_A
DAEFVVRFFLRAAKLIEDPERRRFFLEAARVAAEAANDPELEELVRKVEREL (SEQ ID NO: 9)
MSSSSDFEFVTNFFINAAKKEKDPKKRKFWIENAKVAAKTGNNPELVKKAEEVEKELSSSSLE
(SEQ ID NO: 19)
>HHH_b2_04208_000000083_0001_A
SESHRLFREALRKLLELLEEGDPEKARELFERVLERLKELGNEVAALILEFTYRFN (SEQ ID
NO: 10)
MSSKEAQRIFEEAMKKIIELLKEGKKEEAEKIYKEAVKKLTELGDETGVVILKFTYEFNSSLE
(SEQ ID NO: 20)
>HHH_b2_08356_000000246_0001_A
NALEAMERVIRLLREALRIEDPEEAERVLREAERLARESRSPLLELLVKISLEEL (SEQ ID
NO: 11)
MSSSFLQKMKEVIELVAKANKCKDKEEAEKLLEKALKIAKEADSPLLVKLVEIAKKKKSSSLE
(SEQ ID NO: 21)

In one embodiment, the polypeptide comprises an amino acid sequence at least 75% identical to the amino acid sequence selected from SEQ ID NO:2-21. In another embodiment, the polypeptide comprises an amino acid sequence at least 90% identical to the amino acid sequence selected from SEQ ID NO:2-21. In a further embodiment, the polypeptide comprises an amino acid sequence at least 95% identical to the amino acid sequence selected from SEQ ID NO:2-21.

In one embodiment, relative to the reference amino acid sequence, the polypeptide is identical at 1, 2, 3, 4, 5, 6, 7, 8, or all of the identified residues that form an interface with cortisol and the polypeptide of embodiment of the first aspect of the disclosure. The interface residues are shown in bold font in SEQ ID NO:2-21. In another embodiment, substitutions relative to the reference sequence are conservative amino acid substitutions.

In another embodiment of this second aspect, the disclosure provides fusion proteins comprising the polypeptide of any embodiment of the second aspect fused to one or more functional domains. In various non-limiting embodiments, the functional domain may comprise, for example, a targeting domain, a detectable domain, a scaffold domain, a secretion signal, an Fc domain, or a further therapeutic peptide domain. In one embodiment, the functional domain comprises a first domain of a detectable protein that can be complemented by a second domain of the detectable domain when brought into proximity by, for example, cortisol induced dimerization of a first polypeptide of the first aspect of the disclosure, and a second polypeptide according to this second aspect of the disclosure. In one embodiment, the first domain and second domains of the detectable protein comprise first and second domains of a luciferase protein. The functional domain(s) may be present as an insertion at a loop region of the polypeptides, and/or at one or both termini of the fusion protein.

In another aspect the disclosure provides nucleic acids encoding the polypeptide or fusion protein of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded peptide or chimeric molecular construct, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptide or fusion protein of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptide, fusion protein, nucleic acid or expression vector (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

The disclosure also provides pharmaceutical compositions, comprising:
(a) the polypeptide, fusion protein, nucleic acid, expression vector, and/or host cell of any embodiment or combination of embodiments herein; and
(b) a pharmaceutically acceptable carrier.

The compositions may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The polypeptide, fusion protein, nucleic acid, expression vector, and/or host cell may be the sole active agent in the composition, or the composition may further comprise one or more other agents suitable for an intended use.

In another embodiment, the disclosure provides kits, comprising
(a) one or more first polypeptides or fusion proteins according to any embodiment or combination of embodiments of the first aspect of the disclosure, or a nucleic acid/expression vector encoding the first polypeptide or fusion protein; and
(b) one or more second polypeptides or fusion proteins according to any embodiment or combination of embodiments of the second aspect of the disclosure, or a nucleic acid/expression vector encoding the second polypeptide or fusion protein.

The kits of this aspect can be used as a cortisol biosensor, as described herein.

In one embodiment,
(i) the one or more first polypeptides or fusion proteins comprise a fusion protein of the first aspect of the disclosure, wherein the one or more functional domains comprises a first domain of a detectable protein; and
(ii) the one or more second polypeptides or fusion proteins comprise a fusion protein of the second aspect of the disclosure, wherein the one or more functional domains comprises a second domain of the detectable protein;
wherein the first domain of the detectable protein and the second domain of the detectable protein are only detectable when brought into proximity by formation of a chemically-induced dimer complex comprising cortisol, the first fusion protein, and the second fusion protein.

Any detectable protein may be used as suitable for an intended purpose. In various embodiments, the detectable protein may comprise, but is not limited to, luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease).

In one embodiment, the detectable protein comprises first and second luciferase domains. Exemplary first and second luciferase domains comprise SEQ ID NO:22 and 23.

(SEQ ID NO: 22)
MVFTLEDFVGDWEQTAAYNLDQVLEQGGVSSLLQNLAVSVTPIQRIVRS
GENALKIDIHVIIPYEGLSADQMAQIEEVFKVVYPVDDHHFKVILPYGT
LVIDGVTPNMLNYFGRPYEGIAVFDGKKITVTGTLWNGNKIIDERLITP
DGSMLFRVTINS
(SEQ ID NO: 23)
VTGYRLFEEIL

In one embodiment, the fusion proteins comprise an amino acid sequence at least 50%, 55%, 60%, 65%, 70%, 75%, 80%, 85%, 90%, 95%, 98%, or 100% identical to the amino acid sequence selected from SEQ ID NO:24 and 25. The amino acid sequences of SEQ ID NO:24 and 25 are shown in Table 3.

TABLE 3
H11 = HHH_b2_00727_000000213
>H11-lgBit
MSSASTLVKTFSLCMEALSIEDPEKREEVYEKARKLAEENNDPAALFLVESIKKQHEQSGG
SGGMVFTLEDFVGDWEQTAAYNLDQVLEQGGVSSLLQNLAVSVTPIQRIVRSGENALKIDI
HVIIPYEGLSADQMAQIEEVFKVVYPVDDHHFKVILPYGTLVIDGVTPNMLNYFGRPYEGI
AVFDGKKITVTGTLWNGNKIIDERLITPDGSMLFRVTINS (SEQ ID NO: 24)
mhcy = hcy129.1_CID
>mhcy-smBit
MTSSKEAEEAIRDMLRRWYEAINKGDMEKLKSLVDPDASFHFAITNQQYDKEQFLEMIKEA
LKQDLKVEVKSIHIQQQPRGDHVTVTVHVEAHMNQNGQTHTFTVTDHYHFVRKGDSWKITR
TQWHIHLQGGSGSGGSGGGGVTGYRLFEEIL (SEQ ID NO: 25)

In other embodiments of the kit of any embodiment herein, the kit may further comprise one or more of:
(i) cortisol; and/or
(ii) a means for detection of the reporter protein, including but not limited to a luciferase substrate.

In another aspect, the disclosure provides methods for treating a disorder associated with cortisol, comprising administering to a subject in need thereof an amount effective to treat the disorder of the polypeptide or fusion protein of any embodiment or combination of embodiments of the first aspect of the invention; or a nucleic acid, expression vector, host cell, and/or pharmaceutical composition thereof.

In various embodiments, the disorder is selected from the group consisting of Cushing's syndrome and Addison's disease. Cushing's syndrome is a hormonal disorder caused by prolonged exposure to high levels of cortisol. The cortisol binding polypeptides of the disclosure can be used to reduce cortisol levels, alleviating the symptoms and complications of the disease. Addison's disease is a condition in which the adrenal glands do not produce enough cortisol. The cortisol binding polypeptides of the disclosure can be used to help to manage the symptoms of Addison's disease.

The subject may be any subject that has a relevant disorder or may be at risk of the relevant disorder. In one embodiment, the subject is a mammal, including but not limited to humans, dogs, cats, horses, cattle, etc.

As used herein, an “effective” amount refers to an amount of the polypeptide, fusion protein, nucleic acid, expression vector, host cell, and/or pharmaceutical composition that is effective for treating the disorder. The polypeptides, fusion proteins nucleic acids, expression vectors, and/or host cells are typically formulated as a pharmaceutical composition, such as those disclosed above, and can be administered via any suitable route, including but not limited to orally, by inhalation spray, ocularly, intravenously, subcutaneously, intraperitoneally, and intravesicularly in dosage unit formulations containing conventional pharmaceutically acceptable carriers, adjuvants, and vehicles.

Any suitable dosage range may be used as determined by attending medical personnel. Dosage regimens can be adjusted to provide the optimum desired response. A suitable dosage range for the polypeptides or fusion proteins may, for instance, be 0.1 ug/kg-100 mg/kg body weight; alternatively, it may be 0.5 ug/kg to 50 mg/kg; 1 μg/kg to 25 mg/kg, or 5 μg/kg to 10 mg/kg body weight. In some embodiments, the recommended dose could be lower than 0.1 mcg/kg, especially if administered locally (such as by intra-tumoral injection). In other embodiments, the recommended dose could be based on weight/m2 (i.e. body surface area), and/or it could be administered at a fixed dose (e.g., 0.05-100 mg). The polypeptides, fusion proteins, nucleic acids, expression vectors, and/or host cells can be delivered in a single bolus, or may be administered more than once (e.g., 2, 3, 4, 5, or more times) as determined by an attending physician.

In another aspect, the disclosure provides methods for detecting cortisol in a biological sample, comprising
(a) contacting the biological sample with an amount of the polypeptide or fusion protein of any embodiment or combination of embodiments of the first aspect of the disclosure effective bind to cortisol in the biological sample to produce a binding complex, and
(b) detecting the binding complex in the biological sample, thereby detecting cortisol in the biological sample.

In another embodiment, the methods comprise
(a) contacting the biological sample with:
   (i) one or more first polypeptides or fusion proteins any embodiment or combination of embodiments of the first aspect of the disclosure; and
   (ii) one or more second polypeptides or fusion proteins any embodiment or combination of embodiments of the second aspect of the disclosure; and
(b) detecting a ternary binding complex in the biological sample between (i) the first polypeptide or fusion protein, (ii) the second polypeptide or fusion protein, and (iii) cortisol present in the biological sample, thereby detecting cortisol in the biological sample.

In one embodiment,
(i) the one or more first polypeptides or fusion proteins comprise a fusion protein, wherein the one or more functional domains comprises a first domain of a detectable protein; and
(ii) the one or more second polypeptides or fusion proteins comprise a fusion protein, wherein the one or more functional domains comprises a second domain of the detectable protein;
wherein the first domain of the detectable protein and the second domain of the detectable protein are only detectable upon formation of the ternary complex.

In another embodiment, the detectable protein may comprise, but is not limited to, luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease). In one embodiment, the detectable protein comprises a luciferase

The biological sample may be any suitable sample, including but not limited to a blood sample. The methods of detection have many applications. In various non-limiting embodiments, the methods may involve:
(i) Diagnosis: Quick assessment of cortisol levels can aid in the diagnosis of adrenal insufficiency, Cushing's syndrome, Addison's disease, or other adrenal disorders. Prompt diagnosis can improve patient outcomes by enabling timely interventions. In these embodiments, the subject may be one at risk of adrenal insufficiency, Cushing's syndrome, or other adrenal disorders. In this embodiment, a low level of binding complexes relative to control indicates the presence of a disorder associated with low cortisol levels (such as Addison's disease), and a high level of binding complexes relative to control indicates the presence of a disorder associated with high cortisol levels (such as Cushing's syndrome).
(ii) Monitoring treatment: Regular monitoring of cortisol levels is essential in the management of patients with adrenal disorders or those receiving corticosteroid therapy. A stick-test (i.e., blood sample) can provide quick results, enabling healthcare providers to adjust treatment plans accordingly. In these embodiments, the subject may be one being treated for an adrenal disorder including but not limited to adrenal insufficiency, Cushing's syndrome, Addison's disease, or other adrenal disorders. In one embodiment, if the monitoring indicates that the treatment is not reducing cortisol levels (for disorders associated with high cortisol levels, such as Cushing Syndrome) or is not increasing cortisol levels (for disorders associated with low cortisol levels, such as Addison's disease), the method may further comprise modifying the treatment, either by modifying a dosage of the therapeutic being administered, or by substituting a new therapeutic.
(iii) Stress assessment: Chronic stress can lead to elevated cortisol levels and adversely impact overall health. A stick-test could allow individuals to monitor their cortisol levels regularly, helping them identify and manage stress better. In these embodiments, the subject may be one at risk or suffering from chronic stress.
(iv) Sports medicine: Cortisol levels can provide insights into an athlete's training load, recovery, and overall stress levels. A stick-test could help coaches and athletes optimize training regimens and minimize the risk of injury or overtraining. In these embodiments, the subject is an athlete.
(v) Veterinary medicine: A cortisol stick-test could be used to assess stress levels or diagnose adrenal disorders in animals, improving animal welfare and guiding treatment plans, as discussed above for human subjects.
(vi) Mental health: Cortisol levels have been linked to various mental health conditions, including depression and anxiety. A stick-test could be used as an adjunct tool in mental health assessments and treatment monitoring. In these embodiments, the subject may be one at risk of a mental health condition, including but not limited to depression or anxiety.

### Examples

Despite transformative advances in protein design with deep learning, the design of small-molecule-binding proteins and sensors for arbitrary ligands remains a grand challenge. Here we combine deep learning and physics-based methods to generate proteins with the Nuclear Transport Factor 2 (NTF2) fold, which we employ to computationally design cortisol binders and chemically-induced dimerization (CID) systems. Biophysical characterization of the designed binders revealed nanomolar to low micromolar binding affinities and atomic-level design accuracy by experimental and AlphaFold™ structures. Our design approach is amenable to the design of chemically-induced dimers (CID), and here we construct a de novo CID system with nanomolar sensitivity for cortisol. This approach serves as a general method to design proteins that bind and sense small-molecules for use in a range of analytical, environmental, and biomedical applications.

The design of small molecule binding proteins with high affinity and specificity is of considerable current interest. For example, biosensors and switches that undergo dimerization upon ligand binding (chemically-induced dimerization (CID)) are broadly useful, but current approaches focus on engineering natural CID systems as general methods are not currently available for designing protein small molecule interactions and linking these to protein association.

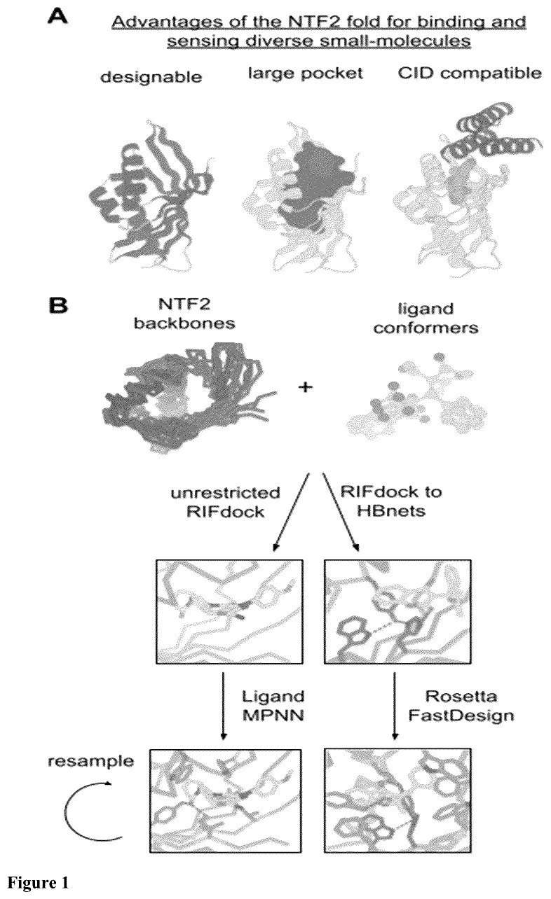

We hypothesized that a more general solution to the small molecule design problem could be attained by combining advances in deep learning based protein fold generation and sequence design. For the former, we reasoned that large sets of scaffolds housing stable pockets could provide the basis for designing binding sites for a wide variety of small molecules, and that the most suitable folds would be both compact (to keep the designs small and modular) and diversifiable (to enable generation of a wide variety of binding sites). For downstream CID applications, we sought a structural solution with the bound ligand sufficiently exposed to enable modulation of a designed protein interaction by ligand binding. Based on these criteria, we chose the compact but readily diversifiable NTF2 fold. For the second, sequence design challenge, we reasoned that the recently developed LigandMPNN could generate more tightly interacting sidechain networks around ligands than previous approaches less able to model natural protein-small molecule interactions (FIG. 1).

### Design and Characterization of a Cortisol-Induced Heterodimer

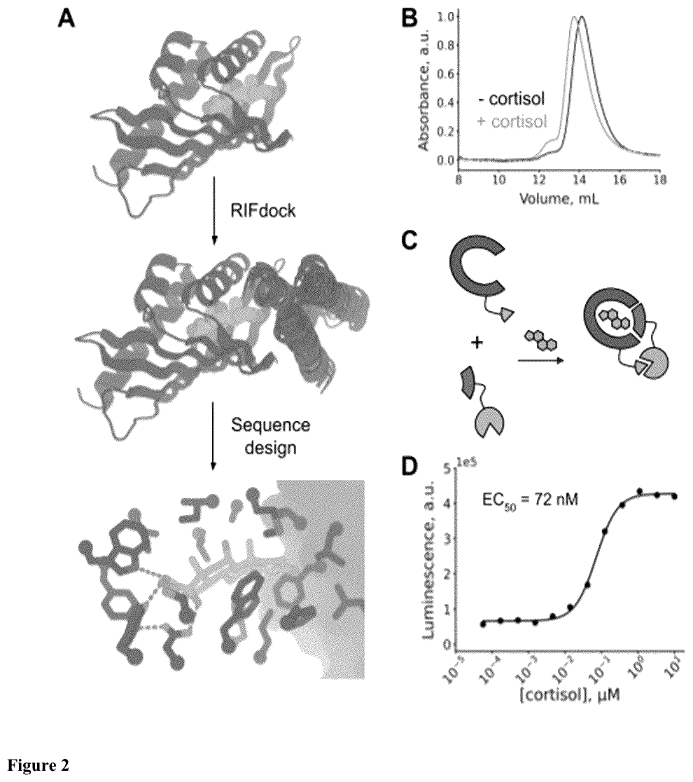

Rational design of a CID system for a user-defined ligand has long been an unsolved problem. With our ability to design protein binders that can recognize cortisol at physiologically relevant concentrations (low nM), we set out to design a cortisol-dependent dimerization system. Since the NTF2 fold leaves part of the small-molecule partially exposed upon binding, we can design protein binders that form a ternary complex that interfaces with the ligand bound state of the NTF2 (FIG. 1A). First, the surface at the opening of the pocket of seq129.1 was redesigned (R43I/R95Q/Q128L) to facilitate docking and design of protein binders to the seq129.1-cortisol complex. After generating this new variant, seq129.1_CID, we performed RIFdock against the seq129.1_CID-cortisol complex with a previously described library of helical bundles (FIG. 2A). This approach generated numerous ternary complexes where both the helical bundle and NTF2 interact with each other and cortisol (FIG. 2A). Sequence design of the resulting docks were carried out with both FastDesign™ and ProteinMPNN™, and the heterodimeric complexes were validated with AlphaFold2™ and filtered for pAE<10 and plddt >85 (FIG. 2A). Selected miniproteins were ordered as synthetic oligonucleotides and cloned into yeast as a library to assess binding to biotinylated seq129.1_CID in the presence or absence of cortisol by FACS. A significant difference was observed in the binding signal of seq129.1_CID when the library was treated with and without cortisol. Populations enriched for binding to seq129.1_CID in the presence of cortisol were collected and sequenced; subsequently we validated the heterodimerization of each hits by FACS in the presence/absence of 100 nM cortisol, which revealed 33 confirmed minibinders that bind to seq129.1_CID only in the presence of cortisol. After identification of putative CIDs by yeast display, we expressed and purified a select subset of these and characterized them in vitro to confirm cortisol-induced dimerization. As an initial test, we combined both seq129.1_CID and the minibinders in the presence or absence of cortisol. For 9 out of 12 selected designs, we observed a clear shift by SEC towards a higher molecular weight species in the presence of cortisol (FIG. 2B). Of these, we analyzed one, mini11, by native mass spectrometry, which revealed a molecular weight for the seq129.1_CID-cortisol-mini11 ternary complex. Together, these data suggest that hits identified by yeast display are cortisol-dependent CIDs.

As a proof-of-concept for the application of the designed CID as a sensor for cortisol, we genetically fused seq129.1_CID and mini11 to the SmBiT and LgBiT components of the NanoBiT™ system, respectively, which reconstitutes NanoBiT™ and generates luminescence when brought in close proximity by a molecular interaction 18. We expressed and purified the fusion constructs from E. coli and when incubated with increasing levels of cortisol, luminescent signal was generated with an estimated EC₅₀ of 25 nM (FIG. 2G). This closely matches the K_{D} of seg129.1 for cortisol, which suggests that a specific interaction between seq129.1_CID and cortisol promotes binding of the minibinder. To assess the affinity of the CID components in the absence of cortisol, we titrated mini11-LgBiT with increasing concentrations of seq129.1_CID-SmBiT, which revealed an estimated K_{D} of ˜5 M, approximately 2 orders of magnitude greater than the EC₅₀ identified for cortisol-induced dimerization, suggesting that the dimerization observed at lower concentrations is dependent on the presence of cortisol. Taken together, these data demonstrate that NTF2-based small-molecule binders designed in this study can be readily engineered to serve as sensors for small-molecules of interest.

### Conclusion

The small-molecule-binders designed and characterized here demonstrate the versatile utility of the de novo designed NTF2 fold and the ability to rationally design custom CIDs. By using deep learning tools, including trRosetta™ hallucination for backbone generation, ProteinMPNN™ and LigandMPNN™ for sequence design, and AlphaFold™ for filtering, we show that protein families that sample novel sequence and structure space can be generated have great utility for the design of functional proteins. We further demonstrated a modular design approach toward an artificial cortisol-induced heterodimer, leading to a novel small-molecule sensor.

### Methods

### Computational Design of Cortisol-Induced Heterodimers

We employed the GALiganddock seq129.1 complex as our target model. This complex features the R43I/R95Q/Q128L mutations, which were chosen based on our experimental SSM profile of seq129.1. The structure of the triple-mutant (seq129.1_CID) was confirmed using AlphaFold2™, revealing a close resemblance to the seq129.1 structure in terms of both backbone conformation and pocket sidechain geometry. To design minibinders to the NTF2-cortisol interface, we first used PatchDock™ (Cao et al.) to find the initial seeding positions for the miniprotein scaffolds against the target interface, and subsequently created Rotamer interaction field (RIF) for both the exposed pocket residues on NTF2 and the cortisol ligand. The miniprotein library described previously (Bennett et al.) was docked into the field to yield around 5 millions docks. A rapid design step (called the predictor, Cao et al.) was used to rank those in silico designs using Rosetta™ ddG and contact molecular surface in which 1 million docks were selected for the downstream Rosetta design. Next, the interfaces between minibinder, NTF2, and cortisol were optimized by Rosetta FastDesign™ as described previously (Cao et al.) but with cortisol being recognized by Rosetta at the designed interface. All designs were filtered by contact molecular surface >380, contact patch >170, Rosetta ddG <−35 prior to ProteinMPNN™ sequence redesign where residues within 5 Å of the cortisol ligand were fixed. Finally, we ran Alphafold2™ prediction with the initial guess protocol (Bennett et al.) where all designs passing pae_interaction <10 and pLDDT_binder >85 were ordered on a synthetic oligo pool.

### Yeast Display and FACS to Screen for Chemical Induced Dimerization

Yeast surface display library containing 60 k designed minibinders was prepared as previously described 24. After the induction of yeast cells in SGCAA medium supplemented with 0.2% glucose, cells were washed with PBSF and incubated with 1 μM purified biotinylated seq129.1_CID, anti-c-Myc fluorescein isothiocyanate (FITC, Miltenyi Biotech) and streptavidin-phycoerythrin (SAPE, ThermoFisher) in the presence or absence of 1 μM cortisol for 1 h at room temperature. Cell sorting was performed using a Sony SH800S cell sorter with software version 2.1.5. 3 million of cells circled in the red region of FACS 2D-plot were collected and streaked on agar plates. 96 colonies were randomly picked from these plates and cultured in C-Trp-Ura media, followed by induction in SGCAA media. The yeast cells of each clone were divided into two groups: one group was incubated with 0.2 μM biotinylated seq129.1_CID/anti-c-Myc-FITC/SAPE while the other group was treated with 0.2 μM biotinylated seq129.1_CID/anti-c-Myc-FITC/SAPE along with 0.2 μM cortisol. All cells were then analyzed with an Invitrogen Attune flow cytometer. 40 out of 96 clones exhibited substantial population shifts in the presence of cortisol when compared to the group without cortisol on the FACS 2D-plots. 12 of them were sequenced and selected for expression in E. coli for downstream biochemical characterization.

### Characterization of Cortisol-Induced Dimerization by SEC

An N-terminal AviTag construct of seq129.1_CID and C-terminal his-tag-containing construct of mini11 as described above. The two protein components of the CID, Nterm-AviTag-seq129.1_CID and mini11-HHHHHH (SEQ ID NO: 26), were incubated at 1 μM in the presence or absence of 10 μM cortisol, incubated for −2 hours at room temperature, and injected onto an S75 increase 10/300 column with a running buffer of 20 mM HEPES, 50 mM NaCl, pH 7.4. Absorbance was monitored at 280 nm over the course of the elution and resulting elution profiles were overlaid to assess potential cortisol-induced shifts in elution.

Characterization of Cortisol-Induced Dimerization and Cortisol Sensing with NanoBiT Fusions

seq129.1_CID and mini11 were genetically fused to SmBiT and LgBiT, respectively, and ordered as eblocks from IDT. Synthetic genes were cloned into pET29b plasmid and transformed into BL21(DE3) and purified as described above. After purification of the CID sensor components, they were mixed together at 1 μM each and then titrated with variable concentrations of cortisol and incubated for 2-3 hours, after which the luciferase substrate diphenylterazine (DTZ) was added at a final concentration of 25 μM. Immediately after adding DTZ, the luminescent signal was measured on a Neo2 plate reader. To estimate the K_{D} of the dimer by NanoBiT™, the mini11-LgBiT component was kept fixed at 0.1 μM and seq129.1_CID-SmBiT titrated at variable concentrations.

### REFERENCES

1. Jumper, J. et al. Highly accurate protein structure prediction with AlphaFold. Nature 596, 583-589 (2021).
2. Baek, M. et al. Accurate prediction of protein structures and interactions using a three-track neural network. Science 373, 871-876 (2021).
3. Watson, J. L. et al. De novo design of protein structure and function with RFdiffusion. Nature 620, 1089-1100 (2023).
4. Dauparas, J. et al. Robust deep learning-based protein sequence design using ProteinMPNN. Science 378, 49-56 (2022).
5. Glasgow, A. A. et al. Computational design of a modular protein sense-response system. Science 366, 1024-1028 (2019).
6. Tinberg, C. E. et al. Computational design of ligand-binding proteins with high affinity and selectivity. Nature 501, 212-216 (2013).
7. Bick, M. J. et al. Computational design of environmental sensors for the potent opioid fentanyl. Elife 6, (2017).
8. Beltran, J. et al. Rapid biosensor development using plant hormone receptors as reprogrammable scaffolds. Nat. Biotechnol. 40, 1855-1861 (2022).
9. Dou, J. et al. De novo design of a fluorescence-activating β-barrel. Nature 561, 485-491 (2018).
10. Polizzi, N. F. et al. De novo design of a hyperstable non-natural protein-ligand complex with sub-A accuracy. Nat. Chem. 9, 1157-1164 (2017).
11. Polizzi, N. F. & DeGrado, W. F. A defined structural unit enables de novo design of small-molecule-binding proteins. Science 369, 1227-1233 (2020).
12. Basanta, B. et al. An enumerative algorithm for de novo design of proteins with diverse pocket structures. Proc. Natl. Acad. Sci. U.S.A. 117, 22135-22145 (2020).
13. Yeh, A. H.-W. et al. De novo design of luciferases using deep learning. Nature 614, 774-780 (2023).
14. Pan, X. & Kortemme, T. De novo protein fold families expand the designable ligand binding site space. PLoS Comput. Biol. 17, e1009620 (2021).
15. Dou, J. et al. Sampling and energy evaluation challenges in ligand binding protein design. Protein Sci. 26, 2426-2437 (2017).
16. Marcos, E. et al. Principles for designing proteins with cavities formed by curved 1 sheets. Science 355, 201-206 (2017).
17. Wicky, B. I. M. et al. Hallucinating symmetric protein assemblies. Science 378, 56-61 (2022).
18. Dixon, A. S. et al. NanoLuc Complementation Reporter Optimized for Accurate Measurement of Protein Interactions in Cells. ACS Chem. Biol. 11, 400-408 (2016).
19. Bannwarth, C. et al. Extended tight-binding quantum chemistry methods. Wiley Interdiscip. Rev. Comput. Mol. Sci. 11, (2021).
20. Cole, J. C., Korb, O., McCabe, P., Read, M. G. & Taylor, R. Knowledge-Based Conformer Generation Using the Cambridge Structural Database. J. Chem. Inf. Model. 58, 615-629 (2018).
21. Klein, J. C. et al. Multiplex pairwise assembly of array-derived DNA oligonucleotides. Nucleic Acids Res. 44, e43 (2016).
22. Kabsch, W. XDS. Acta Crystallogr. D Biol. Crystallogr. 66, 125-132 (2010).
23. Liebschner, D. et al. Macromolecular structure determination using X-rays, neutrons and electrons: recent developments in Phenix. Acta Crystallogr D Struct Biol 75, 861-877 (2019).
24. Cao, L. et al. Design of protein-binding proteins from the target structure alone. Nature 605, 551-560 (2022).

We claim:

1. A polypeptide comprising an amino acid sequence at least 50% identical to the amino acid sequence of SEQ ID NO:1, wherein, relative to SEQ ID NO:1, residue 43 is I, residue 95 is Q, and residue 128 is L.

2. The polypeptide of claim 1, wherein substitutions relative to SEQ ID NO:1 are selected from the residues shown in the substitution column on Table 1.

3. The polypeptide of claim 1, wherein, relative to SEQ ID NO:1, the polypeptide is identical at 1, 2, 3, 4, 5, 6, 7, 8, 9, 10, 11, 12, 13, 14, or all 15 of the identified residues that interact with cortisol.

4. The polypeptide of claim 1, wherein substitutions relative to the SEQ ID NO:1 are conservative amino acid substitutions.

5. A fusion protein comprising the polypeptide of claim 1 fused to one or more functional domains.

6. A polypeptide comprising an amino acid sequence at least 50% identical to the amino acid sequence selected from the group consisting of SEQ ID NO:2-21.

7. The polypeptide of claim 6, wherein, relative to the reference amino acid sequence, the polypeptide is identical at 1, 2, 3, 4, 5, 6, 7, 8, or all of the identified residues that form an interface with (a) cortisol and (b) a polypeptide comprising an amino acid sequence at least 50% identical to the amino acid sequence of SEQ ID NO:1, wherein, relative to SEQ ID NO:1, residue 43 is I, residue 95 is Q, and residue 128 is L.

8. The polypeptide of claim 6, wherein substitutions relative to the reference sequence are conservative amino acid substitutions.

9. A fusion protein comprising the polypeptide of claim 6 fused to one or more functional domains.

10. A nucleic acid encoding the polypeptide of claim 1.

11. An expression vector comprising the nucleic acid of claim 10 operatively linked to a suitable control sequence.

12. A host cell comprising the expression vector of claim 11.

13. A pharmaceutical composition, comprising:
(a) the polypeptide of claim 1; and
(b) a pharmaceutically acceptable carrier.

14. A kit, comprising:
(a) one or more first polypeptides of claim 1, or a nucleic acid encoding the one or more first polypeptides; and
(b) one or more second polypeptides comprising an amino acid sequence at least 50%, identical to the amino acid sequence selected from SEQ ID NO:2-21, or a nucleic acid encoding the one or more second polypeptides.

15. A method for treating a disorder associated with cortisol, comprising administering to a subject in need thereof an amount effective to treat the disorder of the polypeptide of claim 1.

16. A method for detecting cortisol in a biological sample, comprising
(a) contacting the biological sample with an amount of the polypeptide of claim 1 effective bind to cortisol in the biological sample to produce a binding complex, and
(b) detecting the binding complex in the biological sample, thereby detecting cortisol in the biological sample.

17. A method for detecting cortisol in a biological sample, comprising
(a) contacting the biological sample with:
(i) one or more first polypeptides of claim 1; and
(ii) one or more second polypeptides comprising an amino acid sequence at least 50%, identical to the amino acid sequence selected from SEQ ID NO:2-21; and
(b) detecting a ternary binding complex in the biological sample between (i) the first polypeptide or fusion protein, (ii) the second polypeptide or fusion protein, and (iii) cortisol present in the biological sample, thereby detecting cortisol in the biological sample.

18. The method of claim 17, wherein the detectable protein is selected from the group consisting of, bioluminescence resonance energy transfer (BRET) reporters, bimolecular fluorescence complementation (BiFC) reporters, fluorescence resonance energy transfer (FRET) reporters, colorimetry reporters, cell survival reporters, electrochemical reporters, radioactive reporters, and molecular barcode reporters.


SEQUENCE LISTING (26 sequences)

SEQ ID NO: 1 - protein, 129 aa, synthetic construct
TSSKEAEEAIRDMLRRWYEAINKGDMEKLKSLVDPDASFHFAITNQQYDKEQFLEMIKEALKQDLKVEVKSIHIQQQPRGDHVTVTVHVEAHMNQNGQTHTFTVTDHYHFVRKGDSWKITRTQWHIHLQ

SEQ ID NO: 2 - protein, 56 aa, synthetic construct
SEEARLTLIVFRIAVEFGLSQEDYRRLFELARELLERGISLEEIEKILRKVAEELL

SEQ ID NO: 3 - protein, 56 aa, synthetic construct
DLEARLTLIVFRIAVQFGLSQEDYRRLAELARELLERGISLEEIEKILRKVAEELL

SEQ ID NO: 4 - protein, 52 aa, synthetic construct
SKELLEILLRAAERIEDPKERRFLLEFAVFSAEINGDEELLKRLRELLKKLK

SEQ ID NO: 5 - protein, 55 aa, synthetic construct
DREERLREAVEFLVKLLGLSEEQKEELERLVERLVEEGVDLAQALVILFALAIHL

SEQ ID NO: 6 - protein, 57 aa, synthetic construct
DEEFRRILEEAKKLIKHIPDPELRFFLEFLLRAVERSGNPKLLKALESAVKFVEKRL

SEQ ID NO: 7 - protein, 57 aa, synthetic construct
SEDAEELFRRAEKLEEEGNIKKAQVVATLAWFSAAARKDEELLKRIEELVQRLYRLG

SEQ ID NO: 8 - protein, 55 aa, synthetic construct
TRTLVEVFLLLAQAARIDDPEEREKVLREAERVAKENNDPSAERLVELVERKLRN

SEQ ID NO: 9 - protein, 52 aa, synthetic construct
DAEFVVRFFLRAAKLIEDPERRRFFLEAARVAAEAANDPELEELVRKVEREL

SEQ ID NO: 10 - protein, 56 aa, synthetic construct
SESHRLFREALRKLLELLEEGDPEKARELFERVLERLKELGNEVAALILEFTYRFN

SEQ ID NO: 11 - protein, 55 aa, synthetic construct
NALEAMERVIRLLREALRIEDPEEAERVLREAERLARESRSPLLELLVKISLEEL

SEQ ID NO: 12 - protein, 63 aa, synthetic construct
MSSMDKSKLTLKVFNIAMEYGLNDEQYQQLADFAFKKLEEGKSLEEIEKELREKAKELASSLE

SEQ ID NO: 13 - protein, 63 aa, synthetic construct
MSSMDKARMTFECFLIAMKYGLDDEKYQELCKLAFEMLDQGKSFEEIKKEFEEKAKELKSSLE

SEQ ID NO: 14 - protein, 63 aa, synthetic construct
MSSSSGEELLKKLYEGAQTISDKKQKLFLLNFAKSIAEKHGEEELLKKIKEAIKELQSXSSLE

SEQ ID NO: 15 - protein, 63 aa, synthetic construct
MSSSDREKLKEYADILAEQAGLTEEQLEELKKEVDKLVAEGVPYTSALVQLFLKAVQLSSSLE

SEQ ID NO: 16 - protein, 63 aa, synthetic construct
MSDEELKEVVERAKELIEKITDPELKFFLSFLLKAAEESKKPSMIKQLKEAIDFVEKKQSSLE

SEQ ID NO: 17 - protein, 63 aa, synthetic construct
MSSPEAKKYYEEAKKLAEEGDYKKALVKATLALFLAQLDNDEELVEKTKELLEEIQKKRSSLE

SEQ ID NO: 18 - protein, 63 aa, synthetic construct
MSSASTLVKTFSLCMEALSIEDPEKREEVYEKARKLAEENNDPAALFLVESIKKQHEQSSSLE

SEQ ID NO: 19 - protein, 63 aa, synthetic construct
MSSSSDFEFVTNFFINAAKKEKDPKKRKFWIENAKVAAKTGNNPELVKKAEEVEKELSSSSLE

SEQ ID NO: 20 - protein, 63 aa, synthetic construct
MSSKEAQRIFEEAMKKIIELLKEGKKEEAEKIYKEAVKKLTELGDETGVVILKFTYEFNSSLE

SEQ ID NO: 21 - protein, 63 aa, synthetic construct
MSSSFLQKMKEVIELVAKANKCKDKEEAEKLLEKALKIAKEADSPLLVKLVEIAKKKKSSSLE

SEQ ID NO: 22 - protein, 159 aa, synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 23 - protein, 11 aa, synthetic construct
VTGYRLFEEIL

SEQ ID NO: 24 - protein, 223 aa, synthetic construct
MSSASTLVKTFSLCMEALSIEDPEKREEVYEKARKLAEENNDPAALFLVESIKKQHEQSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 25 - protein, 153 aa, synthetic construct
MTSSKEAEEAIRDMLRRWYEAINKGDMEKLKSLVDPDASFHFAITNQQYDKEQFLEMIKEALKQDLKVEVKSIHIQQQPRGDHVTVTVHVEAHMNQNGQTHTFTVTDHYHFVRKGDSWKITRTQWHIHLQGGSGSGGSGGGGVTGYRLFEEIL

SEQ ID NO: 26 - protein, 6 aa, synthetic construct
HHHHHH